(19) United States
(12) Patent Application Publication
(10) Pub. No.: US 2012/0053235 A1
(43) Pub. Date: Mar. 1, 2012

(54) Dihydromyricetin as an IKK-beta inhibitor used for treatment of arthritis, cancer and autoimmune conditions, and other diseases or disorders

(75) Inventors: Liang LIU, Hong Kong (HK); Hua ZHOU, Hong Kong (HK); Ting LI, Hong Kong (HK); Kam Wai WONG, Hong Kong (HK); Zhi Hong JIANG, Hong Kong (HK)

(73) Assignee: HONG KONG BAPTIST UNIVERSITY, Hong Kong (HK)

(21) Appl. No.: 13/197,768

(22) Filed: Aug. 3, 2011

Related U.S. Application Data
(60) Provisional application No. 61/377,992, filed on Aug. 30, 2010.

(51) Int. Cl. A61K 31/35; A61P 29/00; A61P 11/00; A61P 11/06; A61P 35/00; A61P 3/10; A61P 25/28; A61P 19/02; A61P 25/16; A61P 25/14; A61P 37/08; A61P 17/00; A61P 1/04; A61P 19/04; A61P 5/14; A61P 7/02; A61P 7/06; A61P 37/06; A61P 19/06; A61P 17/06; A61P 37/00

(52) U.S. Cl. 514/456

(57) ABSTRACT

Use of dihydromyricetin (DMY) as an NF-κB inhibitor or an IKK-β inhibitor for the treatment of arthritis, cancer, autoimmune conditions and other disease is provided. A pharmaceutical composition comprising DMY is also provided.

### CROSS-REFERENCE TO RELATED APPLICATION

This application claims benefit under 35 U.S.C. §119(e) of U.S. Provisional Application having Ser. No. 61/377,992 filed 30 Aug. 2011, which is hereby incorporated by reference herein in its entirety.

### FIELD OF INVENTION

This invention relates to known compound and its uses for the treatment of autoimmune conditions, rheumatoid arthritis, chronic obstructive pulmonary disease, asthma, cancer, diabetes mellitus, neurodegenerative disease, immunological disorder, hypersensitivity, and arthritis.

### BACKGROUND OF INVENTION

Chronic diseases such as immune-related disorders, including cancers are debilitating and may even be fatal.

### SUMMARY OF INVENTION

It is an object of the present invention to provide new compound for the treatment of immune-related disorders.

The present invention relates to the use of the compound is dihydromyricetin (DMY) of Formula (I) as illustrated in FIG. 1A. It is based on the discovery that DMY is an NF-κB inhibitor.

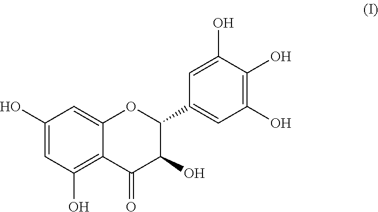

The medicinal herb, Rattan Tea, known as the tender stems and leaves of Amplopsis grossedentata, has been popularly used in South China for medicinal usages. Studies have showed that DMY, characterized as a flavonoid, is the major bioactive constituent of A. grossedentata.

In one aspect, the present invention provides a method of treating auto-immune disease, rheumatoid arthritis, chronic obstructive pulmonary disease (COPD), asthma, cancer, diabetes mellitus, neurodegenerative disease, immunological disorder, and arthritic disorder comprising administrating a therapeutically effective amount of DMY.

In an exemplary embodiment, the aforesaid treatment is effected via the inhibition of T cell proliferation and/or T cell activation, NF-κB and/or IκB kinase β (IKK-β) activation, and AP-1 activation.

In another exemplary embodiment, DMY inhibits NF-κB activation by its inhibitory action on activity of IKK-β; in a further exemplary embodiment, the inhibitory action on IKK-1 is conducted by the direct binding of DMY to IKK-β on novel binding site(s).

In another exemplary embodiment, DMY inhibits NF-κB activation by suppressing mitogen-activated protein kinase (MAPK) pathway, and in a further exemplary embodiment, DMY inhibits phosphorylation of p38 kinase and c-Jun N-terminal kinase (JNK) to suppress MAPK pathway.

In another exemplary embodiment, the neurodegenerative diseases may be Alzheimer's disease, Parkinson's disease, amyotrophic lateral sclerosis, ataxia telangiectasia, spinocerebellar atrophy, multiple sclerosis, or Huntington's chorea.

In another exemplary embodiment, the immunological disorders may be allergic rhinitis, allergic dermatitis, allergic contact dermatitis, allergic shock, asthma, papular urticaria, leucoderma, hypersensitivity vasculitis, hypersensitivity pneumonia, ulcerative colitis, glomerulonephritis, drug rashes, systemic lupus erythematosus, rheumatoid arthritis, scleroderma, multiple sclerosis, hyperthyroidism, idiopathic thrombocytopenic, autoimmune hemolytic anemia, allograft rejection, or hemolytic transfusion reaction.

In another exemplary embodiment, the disease may be arthritic disorders, such as rheumatoid arthritis, ankylosing spondylitis, gout, periarthritis, osteoarthritis, Reiter syndrome, psoriatic arthritis, post-traumatic arthritis, or enteropathic arthritis.

In yet another exemplary embodiment, DMY is administrated at a concentration of 0.1 to 100 mg/kg.

Another aspect of the present invention is a pharmaceutical composition comprising DMY admixed with a pharmaceutical carrier suitable for use by an oral administration. In one exemplary embodiment, as the pharmaceutical carrier may be starch, sugar, lactose, or others suitable therefor.

In an exemplary embodiment, the pharmaceutical composition is administrated to a subject in need thereof for the treatment of a disease or disorder. In one exemplary embodiment, the disease may be auto-immune disease, rheumatoid arthritis, chronic obstructive pulmonary disease (COPD), asthma, cancer, diabetes mellitus, neurodegenerative disease, immunological disorders, or arthritic disorders. In another exemplary embodiment, the neurodegenerative diseases may be Alzheimer's disease, Parkinson's disease, amyotrophic lateral sclerosis, ataxia telangiectasia, spinocerebellar atrophy, multiple sclerosis, or Huntington's chorea. In another exemplary embodiment, the immunological disorders may be allergic rhinitis, allergic dermatitis, allergic contact dermatitis, allergic shock, asthma, papular urticaria, leucoderma, hypersensitivity vasculitis, hypersensitivity pneumonia, ulcerative colitis, glomerulonephritis, drug rashes, systemic lupus erythematosus, rheumatoid arthritis, scleroderma, multiple sclerosis, hyperthyroidism, idiopathic thrombocytopenic, autoimmune hemolytic anemia, allograft rejection, or hemolytic transfusion reaction. In another exemplary embodiment, the arthritic disorders may be rheumatoid arthritis, ankylosing spondylitis, gout, periarthritis, osteoarthritis, Reiter syndrome, psoriatic arthritis, post-traumatic arthritis, or enteropathic arthritis.

In yet another embodiment, the pharmaceutical composition is in a form suitable for topical or oral use.

In accordance with a further aspect of this invention, a method of treating neurodegenerative disease is provided comprising administrating a therapeutically effective amount of an activated protein 1 (AP-1) inhibitor and/or IKK-β/NF-κB inhibitor to a subject in need thereof, in which the AP-1 inhibitor and/or IKK-β/NF-κB inhibitor is DMY.

In one exemplary embodiment, DMY suppresses AP-1 by suppressing p38 kinase and JNK phosphorylation. In another exemplary embodiment, DMY inhibits the nuclear translocation of c-Fos and c-Jun, the members of AP-1, so as to suppress AP-1. In another exemplary embodiment, DMY inhibits AP-1 by suppressing c-Fos nuclear localization.

In another exemplary embodiment, the neurodegenerative disease may be Alzheimer's disease, Parkinson's disease, amyotrophic lateral sclerosis, ataxia telangiectasia, spinocerebellar atrophy, multiple sclerosis, or Huntington's chorea.

In another aspect, the present invention provides a method for the treatment of immunological disorder comprising administrating a therapeutically effective amount of an immunosuppressive compound to a subject in need thereof, in which the immunosuppressive compound is DMY.

In one exemplary embodiment, DMY suppresses immune system by inhibiting human T-cell proliferation revoked by anti-OKT-3/anti-CD28 and phorbol myristate acetate (PMA)/ionomycin (P/I). In another exemplary embodiment, DMY suppresses immune system by inhibiting interleukin (IL)-2 production in human T-cell mediated by anti-OKT-3/anti-CD28 and phorbol myristate acetate (PMA)/ionomycin (P/I).

In another exemplary embodiment, the immunological disorder may be allergic rhinitis, allergic dermatitis, allergic contact dermatitis, allergic shock, asthma, papular urticaria, leucoderma, hypersensitivity vasculitis, hypersensitivity pneumonia, ulcerative colitis, glomerulonephritis, drug rashes, systemic lupus erythematosus, rheumatoid arthritis, scleroderma, multiple sclerosis, hyperthyroidism, idiopathic thrombocytopenic, autoimmune hemolytic anemia, allograft rejection, or hemolytic transfusion reaction.

The invention according to another aspect provides a method of treating an arthritic disorder comprising administrating a therapeutically effective amount of an arthritic inhibitor to a subject in need thereof, in which the arthritic inhibitor is DMY.

In another exemplary embodiment, the arthritic disorder may be rheumatoid arthritis, ankylosing spondylitis, gout, periarthritis, osteoarthritis, Reiter syndrome, psoriatic arthritis, post-traumatic arthritis, or enteropathic arthritis.

In one aspect, the present invention provides a NF-κB inhibitory compound in which the compound is DMY. In one exemplary embodiment, the NF-κB inhibitory compound can be used for the treatment of auto-immune disease, rheumatoid arthritis, chronic obstructive pulmonary disease (COPD), asthma, cancer, diabetes mellitus, neurodegenerative disease, immunological disorders, and arthritic disorders.

In another aspect of the present invention, an IKK-β inhibitory compound is provided in which the compound is DMY. In an exemplary embodiment, DMY inhibits activity of IKK-β by its direct binding to IKK-β. In one exemplary embodiment, the NF-κB inhibitory compound can be used for the treatment of auto-immune disease, rheumatoid arthritis, chronic obstructive pulmonary disease (COPD), asthma, cancer, diabetes mellitus, neurodegenerative disease, immunological disorders, and arthritic disorders.

In yet another aspect of the present invention, a use of DMY as an inhibitor of NF-κB is provided; in one exemplary embodiment, DMY is an inhibitor of IKK-β. In another exemplary embodiment, DMY as an inhibitor of NF-κB or an inhibitor of IKK-β is administrated into a subject in need thereof for the treatment.

In a further aspect, the present invention provides an AP-1 and/or IKK-β/NF-κB suppressive compound is provided in which the compound is DMY. In one exemplary embodiment, the AP-1 and/or IKK-β/NF-κB suppressive compound can be used for the treatment of neurodegenerative disease such as Alzheimer's disease, Parkinson's disease, amyotrophic lateral sclerosis, ataxia telangiectasia, spinocerebellar atrophy, multiple sclerosis, or Huntington's chorea.

The present invention in yet another aspect provides an immunosuppressive compound in which the compound is DMY. In one exemplary embodiment, the immunosuppressive compound can be used for the treatment of the immunological disorder such as allergic rhinitis, allergic dermatitis, allergic contact dermatitis, allergic shock, asthma, papular urticaria, leucoderma, hypersensitivity vasculitis, hypersensitivity pneumonia, ulcerative colitis, glomerulonephritis, drug rashes, systemic lupus erythematosus, rheumatoid arthritis, scleroderma, multiple sclerosis, hyperthyroidism, idiopathic thrombocytopenic, autoimmune hemolytic anemia, allograft rejection, or hemolytic transfusion reaction.

In another aspect of the present invention, an anti-arthritic compound is provided in which the compound is DMY. In one exemplary embodiment, the immunosuppressive compound can be used for the treatment of arthritic disorder such as rheumatoid arthritis, ankylosing spondylitis, gout, periarthritis, osteoarthritis, Reiter syndrome, psoriatic arthritis, post-traumatic arthritis, or enteropathic arthritis.

### BRIEF DESCRIPTION OF FIGURES

FIG. 1 illustrates the chemical structure of DMY according to an embodiment of the present invention.

FIGS. 2A to 2B show the results of a study of DMY on its inhibition of T-cell proliferation revoked by anti-OKT-3/anti-CD28 (FIG. 2A) and phorbol myristate acetate (PMA)/ionomycin (P/I) (FIG. 2B) according to one embodiment of the present invention.

FIGS. 3A to 3B show the results of a study of DMY on its inhibition of IL-2 production mediated by anti-OKT-3/anti-CD28 (FIG. 3A) and PMA/ionomycin (P/I) (FIG. 3B) according to one embodiment of the present invention. (In these figures, *** denotes p<0.001; ** denotes p<0.01; and * denotes p<0.05.)

FIGS. 4A to 4B show the results of a study of DMY on its inhibition of the activity of IKK-β (FIG. 4A) and IKK-β phosphorylation (FIG. 4B) according to one embodiment of the present invention. (In these figures, *** denotes p<0.001; ** denotes p<0.01; and * denotes p<0.05.)

FIGS. 5A to 5C show the results of a study of DMY on its competitive activity with biotin-DMY on directly binding with recombinant IKK-β on different binding sites according to one embodiment of the present invention.

FIGS. 6A to 6B show the results of a study of DMY on its suppression of NF-κB nuclear translocation (FIG. 6A) and NF-κB activity (FIG. 6B) according to one embodiment of the present invention. (In these figures, *** denotes p<0.001; ** denotes p<0.01; and * denotes p<0.05.)

FIG. 7 shows the results of a study of DMY on its suppression of p38 and JNK phosphorylation, its suppression of c-Fos and c-Jun nuclear translocation, and its suppression of c-Fos nuclear translocation according to one embodiment of the present invention.

FIGS. 8A to 8D show the results of a study of DMY on its suppression of c-Fos nuclear localization according to one embodiment of the present invention. FIG. 8A demonstrates the result of the control experiment. Content in FIGS. 8B, C, and D are PMA/ionomycin (P/I), DMY in 50 μM with PMA/ionomycin (P/I), and DMY in 100 M with PMA/ionomycin (P/I) respectively.

FIGS. 9A to 9D show the results of a study of DMY on its effect on ear edema induced by dinitrofluorobenzene according to one embodiment of the present invention.

FIGS. 10A to 10D show the results of a study of DMY on its effect on arthritis model induced by collagen II according to one embodiment of the present invention.

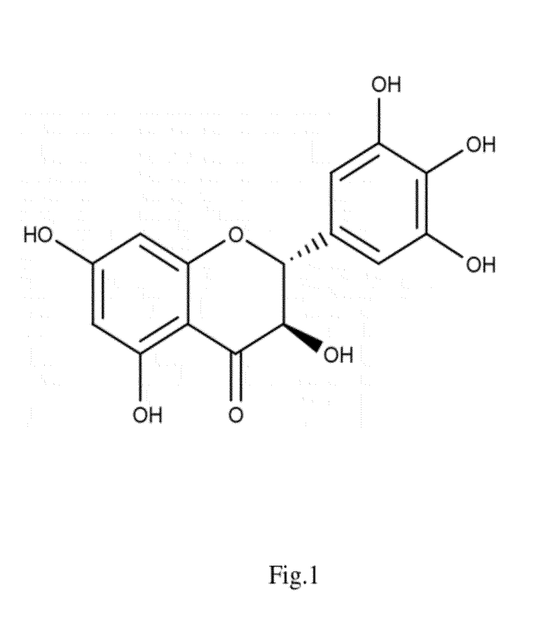

### DETAILED DESCRIPTION OF THE PREFERRED EMBODIMENTS

As used herein and in the claims, “comprising” means including the following elements but not excluding others. When interpreting each statement in this specification that includes the term “comprising”, features other than that or those prefaced by the term may also be present. Related terms such as “comprise” and “comprises” are to be interpreted in the same manner.

Since IKK-β plays a vital role in the regulation of NF-κB signaling pathway which in turn leads to the regulation of transcription of genes involved in important mechanisms within cells such as T-cell activation, the medicinal usages thereof have been widely studied and published. For instance, IKK-β inhibitors have been proven to treat auto-immune diseases [Refs. 1-2], rheumatoid arthritis [Refs. 3-12], chronic obstructive pulmonary disease (COPD) and asthma [Refs. 11-27], cancer [Refs. 28-38], and diabetes [Refs. 39-42]. The references cited for each of the foregoing and hereinafter diseases in square bracket with “[Refs.xx]” with xx referring to the number of the corresponding literatures on the “References” list.

It is part of the present invention that DMY has been found to be inhibitors of IKK-β and NF-κB. As such, it can be deduced by one skilled in the art that DMY as disclosed in the present application can be used for the treatment for the diseases described above as these diseases are associated with the activation of IKK-β and NF-κB.

The activated protein (AP)-1, which is composed of c-Fos and c-Jun and can be activated by p38 and JNK, plays important role in neurodegenerative diseases [Ref. 43].

In addition, NF-κB activation could mediate the Abeta-associated phenotype in Alzheimer disease, suggests the critical role in neurodegenerative diseases [Ref. 44]

It is also part of the present invention that DMY has been found to be suppressor of AP-1 activation and/or IKK-β/NF-κB activation. As such, it can be deduced by one skilled in the art that DMY as disclosed in the present application can be used for the treatment for the diseases described above as these diseases are associated with the activation of AP-1 and NF-κB signaling.

Further, it is part of the present invention that DMY has been found to be suppressor of immune reaction and hypersensitivity. As such, it can be deduced by one skilled in the art that DMY as disclosed in the present application can be used for the treatment for the diseases described above as these diseases are associated with the activation of immune reaction and hypersensitivity.

It is part of the present invention that DMY has been found to be inhibitor of arthritis. As such, it can be deduced by one skilled in the art that DMY as disclosed in the present application can be used for the treatment for the diseases described above as these diseases are associated with arthritis.

The present invention is further defined by the following examples, which are not intended to limit the present invention. Reasonable variations, such as those understood by reasonable artisans, can be made without departing from the scope of the present invention.

### Example 1

### Study on Inhibition of T Cell Proliferation and IL-2 Production

This example describes two assays of T cell proliferation and IL-2 secretion to demonstrate the inhibitory ability of DMY on T cell activation and IL-2 production.

1.1 T Cells Proliferation Assay

The isolated human T lymphocytes (1×10⁵ cells/well) were stimulated with anti-OKT 3/anti-CD28 antibodies or PMA/ionomycin (P/I) in the presence or absence of DMY for 72 hours.

5-bromo-2′-deoxy-uridine (BrdU, Roche) was added to the cells at the 14th hour before the end of stimulation and it could be incorporated into the DNA of the growing cells during the labeling period. The amount of BrdU incorporated into DNA was quantified as an indicator of cell proliferation. BrdU was determined by ELISA according to manufacturer's instructions.

1.2 Enzyme-Linked Immunosorbent Assay (ELISA)

The cells (1×10⁵/well) were incubated in the presence or absence of DMY for 2 hours at the indicated concentrations, and then the cells were stimulated with P/I or anti-OKT-3/anti-CD28 for 48 hours. The cell-free culture supernatants were collected, and then the concentration of IL-2 in the supernatants was determined by ELISA method.

1.3 Results

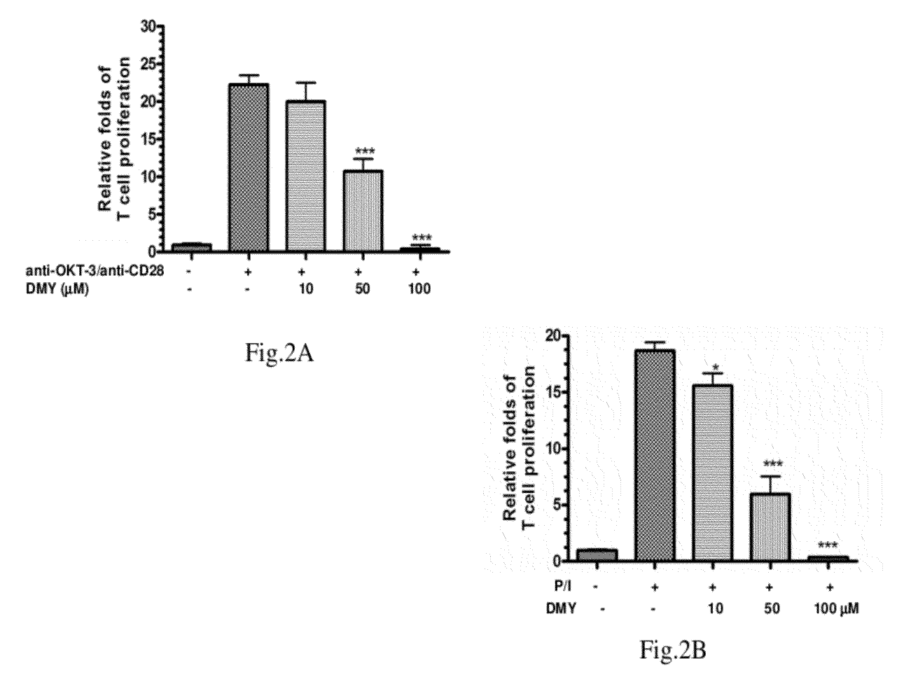

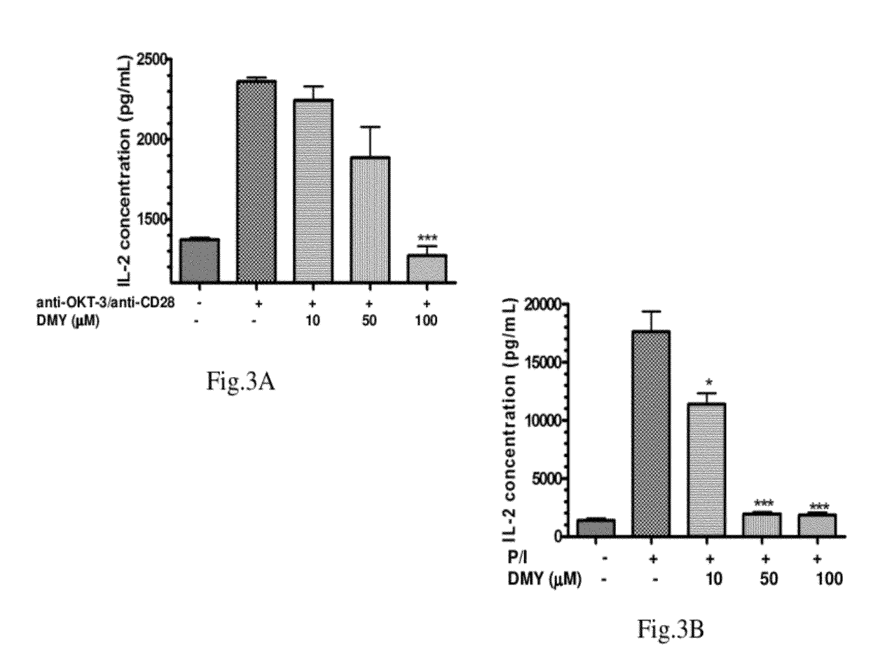

It can be seen from FIGS. 2A and 2B that DMY was shown to block T cell proliferation. DMY (studied at three concentrations of 10, 50, and 100 M) was also illustrated to suppress the production of IL-2 from FIGS. 3A and 3B. Thus in accordance with the present invention, DMY is also shown in FIGS. 2A and 2B to have specific medical uses for the treatment of a disease or disorder such as auto-immune disease, rheumatoid arthritis, chronic obstructive pulmonary disease (COPD), asthma, cancer, diabetes mellitus, neurodegenerative disease; or a disorder such as immunological disorders and arthritic disorders.

### Example 2

### Study on Inhibition of IKK-β Activity and IKK-βPhosphorylation

This example describes an assay that DMY is potent in directly inhibiting IKK-β activity and IKK-α/β phosphorylation.

2.1 IKK Activity Assay

The direct inhibitory effect of DMY on IKK activity was examined by using K-LISA™ IKK-β-Inhibitor Screening Kit (Calbiochem). Both the glutathione-S-transferase (GST)-IκB-α substrate and His-tagged recombinant human IKK-β were incubated with or without DMY in the wells of a glutathione-coated 96-well plate. The reaction was terminated with the addition of kinase stop solution after being incubated at 30° C. for 30 minutes. Then, anti-Phospho IκB-α (Ser32/Ser36) antibody was used to determine the phosphorylated GST-IκB-α substrate, and the horse radish peroxidase (HRP)-conjugated color was developed by 3,3′,5,5′-tetramethylbenzidine (TMB) substrate. ELISA stop solution was used to stop the color development and the absorbance was measured at 450 nm the wavelength at which was directly related to the level of IKK activity.

2.2 Measurement of the Phosphorylation of IKK α/β

Human T lymphocytes (4×10⁶/well) were pretreated with DMY in different concentrations for 60 minutes and then the cells were stimulated with PMA/ionomycin for 30 minutes.

The whole-cell lysates were prepared by lysing the harvested T cells with lysis buffer (50 mM Tris-HCl, pH 7.5, 250 mM NaCl, 5 mM EDTA, 1 mM DTT, 1% Triton, 50 mM NaF, 1 mM sodium orthovanadate, 0.5 mM PMSF and 1× protease inhibitor mix, Roche). Protein concentrations were determined by using Bio-Rad Protein assay (Bio-Rad Laboratories, Inc. Hercules, Calif.). Equal amount of nuclear proteins or whole-cell lysates were analyzed by 10% SDS-polyacrylamide gel electrophoresis (SDS-PAGE). After electrophoresis, the proteins were electro-transferred to the nitrocellulose membranes. After proteins were transferred, the membranes were blocked by 5% dried milk for 60 minutes and then washed three times for 5 minutes in washing interval with TBS-T (Tween-20, 0.05%). The membranes were then incubated with phosphorylation-IKK-α/β primary antibodies overnight at 4° C. and then washed three times with TBS-T. Afterwards, the membranes were incubated again with HRP-conjugated secondary antibodies for 60 minutes. The blots were developed using the enhanced chemiluminescence (ECL, Amersham Bioscience).

2.3 Results

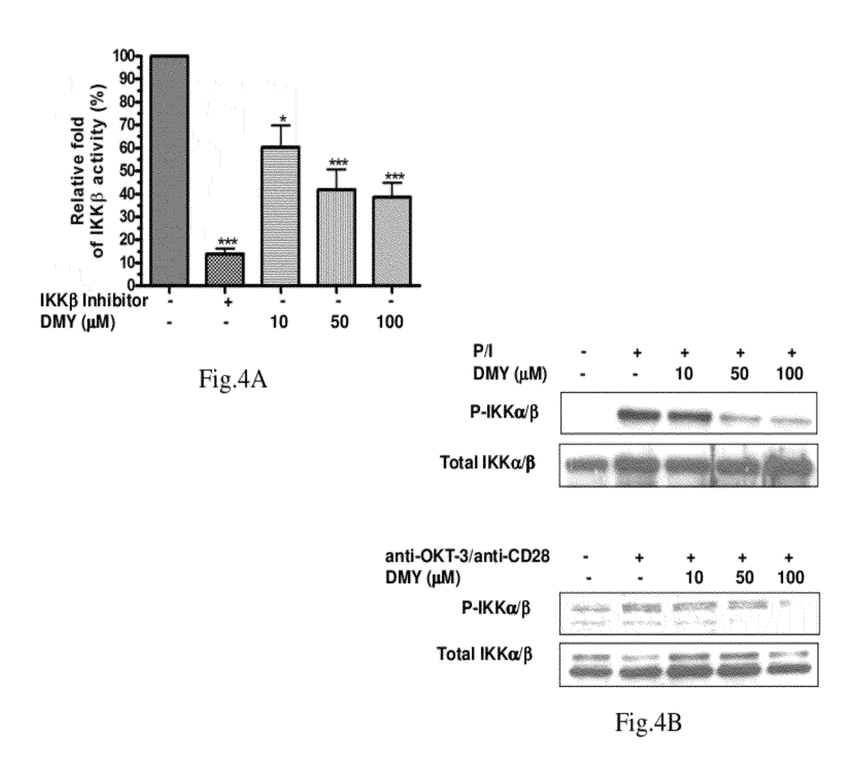

It can be seen from FIG. 4A that DMY at 50 μM and 100 μM significantly inhibited the activity of IKK-β. The IKK-β protein has been proven as a potent kinase for activation of cell signaling pathways that would potentiate inflammation in many inflammatory and autoimmune conditions. Inhibition of IKK-β has thus become the most important strategy for drug discovery in anti-inflammation and anti-cancer. Consequently, DMY was proved from this study that it can be used in treating inflammation and cancer.

In addition, from FIG. 4B, DMY was shown to inhibit IKK-ca/3 phosphorylation mediated by PMA/ionomycin or anti-OKT-3/anti-CD28.

### Example 3

### Study on IKK-β Binding

This example describes the assays to show that DMY can directly bind to IKK-β kinase.

3.1 IKK-β Competition Assay

5 ng of human recombinant IKK-β was incubated with 100 μM of the biotin-DMY in the presence of 0, 1, and 5 folds of concentration of its parental compound. The mixture was dropped on the nitrocellulose membranes, and then detected with streptavidin horseradish peroxidase. The binding signal was then detected by using ECL.

3.2 Binding of DMY-Biotin to IKK-β

Anti-FLAG precipitated from HEK 293 expressing FLAG-IKK-β, FLAG-IKK-β (C179A), FLAG-IKK-β (C662A/C716A) was incubated with 100 μM DMY-biotin, and then the proteins were separated by SDS-PAGE and transferred to nitrocellulose membranes. After blocking with BSA and washing with Phosphate Buffered Saline with Tween-20 (0.05%) (PBS-T), the membranes were incubated with streptavidin horseradish peroxidase (Sigma) and developed with ECL.

3.3 DMY Binding to Novel Site(s) of IKK-β

DMY-biotin was incubated with IKK-β immunoprecipitated from HEK293T cells in presence of DMY, BMS-345541, SC-514 or BOT-64 for 1 hour on ice, and then the proteins were separated by SDS-PAGE and transferred to nitrocellulose membranes. After blocking with BSA and washing with Phosphate Buffered Saline with Tween-20 (0.05%) (PBS-T), the membranes were incubated with steptavidin horseradish peroxidase (Sigma) and developed with ECL. SC-514 presents ATP-competitive and highly selective inhibitor of IKK-β; BMS-345541 presents a selective and allosteric inhibitor of IKK-β; BOT-64 presents the IKK-β inhibitor by targeting the Ser177 and/or Ser181 residues.

3.4 Results

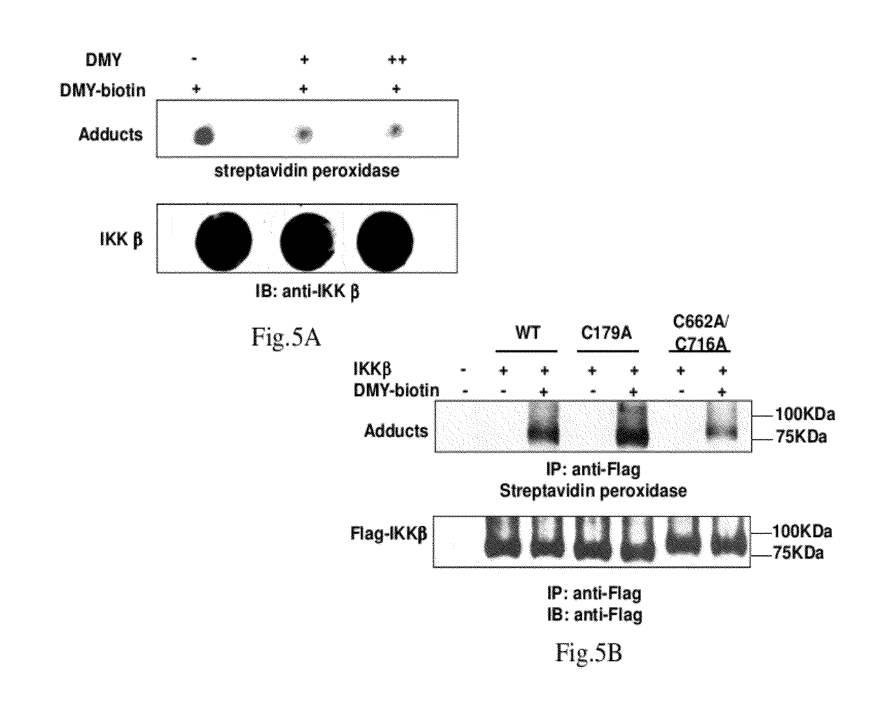

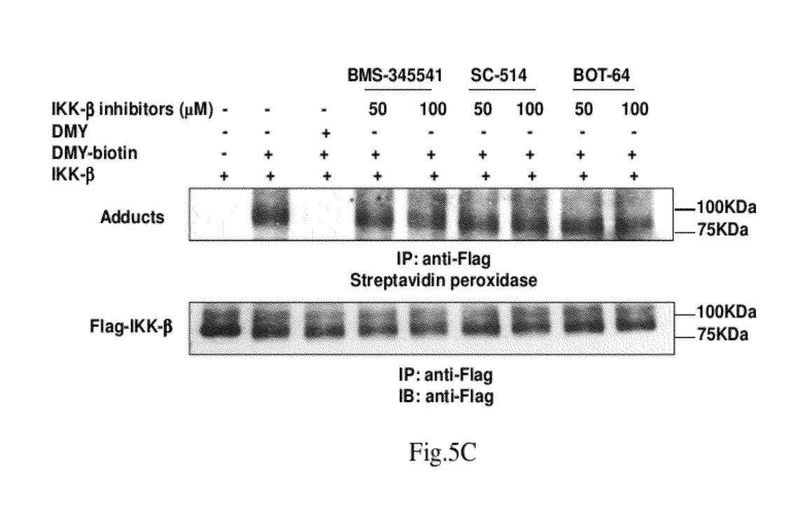

It can be observed from FIG. 5A that the parental compound DMY can compete with biotin-DMY, indicating that the biotin-DMY was confirmed to exhibit an identical binding site(s) as its parental compound DMY. In addition, FIGS. 5B and C showed that biotin-DMY could directly bind to IKK-β on not well-known binding sites, including Cys-179, Cys-662/-716, ATP, allosteric and Ser-177/-181 residues. Hence, DMY was shown to inhibit activity of IKK-β probably via novel binding site(s) on IKK-β protein.

### Example 4

### Study on Suppression of NF-κB Nuclear Translocation and NF-κB Activity

This example describes the assays to show that DMY is effective in inhibiting NF-κB nuclear translocation and NF-κB transcriptional activity in human T cells.

4.1 NF-κβ Nuclear Translocation

Human T lymphocytes (6×10⁶/well) were pretreated with DMY for 1 hour, and subsequently stimulated with 20 ng/ml PMA plus 1 μM ionomycin for 120 minutes. The cells were then harvested and washed with PBS twice. The nuclear proteins of cells were prepared by using NucBuster™ Reagents (Novagen, USA). The washed cells were re-suspended using 60 μl NucBuster™ Reagent for 1 per 301 of packed cells and processed twice by vortexing for 15 seconds, and followed by incubation on ice for 5 minutes and second vortexing for 15 seconds, and finally centrifuged at 16000 g for 5 minutes. The supernatants containing cytoplasmic protein were harvested, and then the cell pellets were re-suspended in 45 μl of NucBuster Extraction Reagent 2. The same vortexing, icing, and repeated vortexing procedures were repeated once to prepare the nuclear proteins of the cells.

Protein concentrations were determined by using Bio-Rad Protein assay. Equal amounts of nuclear proteins or whole-cell lysates were analyzed by 10% SDS-PAGE. After electrophoresis, the proteins were electro-transferred to the nitrocellulose membranes. After proteins were transferred, the membranes were blocked by 5% dried milk for 60 minutes and then washed three times for 5 min in each washing interval with Tris Buffered Saline with Tween 20 (TBS-T). The membranes were incubated with p65 antibodies overnight at 4° C. and then washed three times with TBS-T. Afterwards, the membranes were incubated again with HRP-conjugated secondary antibodies for 60 minutes. The blots were developed using the ECL.

4.2 NF-κβ Reporter Assay

The Jurkat T cells are transiently transfected with NF-κB-Luciferase reporter plasmid with lipofectamine LTX (Invitrogen). The transfected cells were treated with 20 ng/ml PMA plus 1 μM ionomycin in the presence or absence of biotin-DMY for 6 hours. The cells were then lysed in Passive Lysis Buffer (Promega) and the transcriptional activity was determined by measuring the activity of firefly luciferase in a microplate luminometer (Perkin Elmer) using Luciferase Reporter Assay (Promega).

4.3 Results

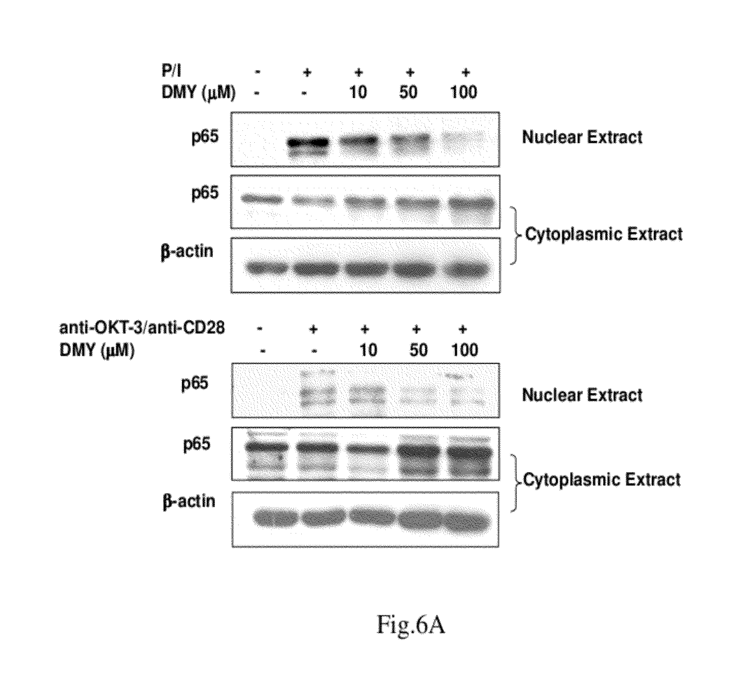

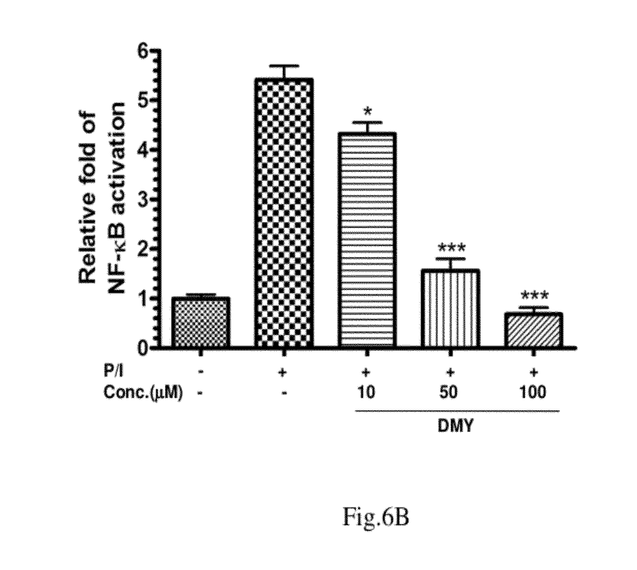

It can be observed respectively from FIGS. 6A and 6B that DMY significantly suppressed NF-κB nuclear translocation and NF-κB transcriptional activity.

### Example 5

Study on Inhibition of JNK and p38 Kinases Phosphorylation, and c-Fos and c-Jun Nuclear Translocation

This example describes the assay to show that DMY inhibits the phosphorylation of JNK and p38 kinases, the nuclear translocation of c-Fos and c-Jun, and the nuclear localization of c-Fos

5.1 Measurement of the Phosphorylation of Mitogen-Activated Protein Kinases (MAPKs) and the Nuclear Translocation of c-Fos and c-Jun

For determination of the phosphorylation of mitogen-activated protein kinases expression, the cells were stimulated by 20 ng/ml PMA plus 1 M ionomycin for 10 minutes. The whole-cell lysates were prepared by lysing the harvested T cells with lysis buffer (50 mM Tris-HCl, pH 7.5, 250 mM NaCl, 5 mM EDTA, 1 mM DTT, 1% Triton, 50 mM NaF, 1 mM sodium orthovanadate, 0.5 mM PMSF and 1× protease inhibitor mix, Roche).

For c-Jun and c-Fos nuclear translocation assays, the human T lymphocytes (6×10⁶/well) were pretreated with DMY for 1 hour, and then stimulated with 20 ng/ml PMA plus 1 M ionomycin for 120 minutes. After the cells were harvested and washed with PBS twice, the nuclear proteins of cells were then prepared using NucBuster™ Reagents. Afterwards, the washed cells were re-suspended using 60 μl NucBuster™ Reagent 1 per 30 μl of packed cells and processed twice by vortexing for 15 seconds, and followed by the incubation on ice for 5 minutes and second vortexing for 15 seconds, and finally centrifuged at 16000 g for 5 minutes. The supernatants containing cytoplasmic proteins were discarded; the cell pellets were re-suspended in 45 μl of NucBuster Extraction Reagent 2. The same vortexing, icing, and repeated vortexing steps were repeated once to prepare the nuclear proteins of the cells.

Protein concentrations were determined by using Bio-Rad Protein assay. Equal amounts of nuclear proteins or whole-cell lysates were analyzed by 10% SDS-PAGE. After electrophoresis, the proteins were electro-transferred to the nitrocellulose membranes. After proteins were transferred, the membranes were blocked by 5% dried milk for 60 minutes and then washed three times for 5 min in each washing interval with TBS-T. The membranes were incubated with corresponding primary antibodies overnight at 4° C. and then washed with three times with TBS-T. Afterwards, the membranes were incubated again with HRP-conjugated secondary antibodies for 60 minutes. The blots were developed using the ECL.

5.2 Examination of the Nuclear Localization of c-Fos

HeLa cells (1×10⁵) were seeded on the 6-well plates with cover slips and cultured overnight. The cells were treated with DMY for 2 hours at 37° C and then stimulated with or without 20 ng/ml PMA plus 1 μM ionomycin for another 2 hours. After stimulation, the cells were fixed with 4% paraformaldehyde for 15 minutes at room temperature and permeabilized by 0.1% Triton X-100 and then stained with phalloidin (Invitrogen) for 3 minutes. The cells were incubated with c-Fos antibody for 2 hour after being stained with phalloidin, and the cells were then incubated with FITC-linked secondary antibody for 1 hour after being washed with PBS for 3 times. The slides were dried in air and mounted onto the glass slides.

5.3 Results

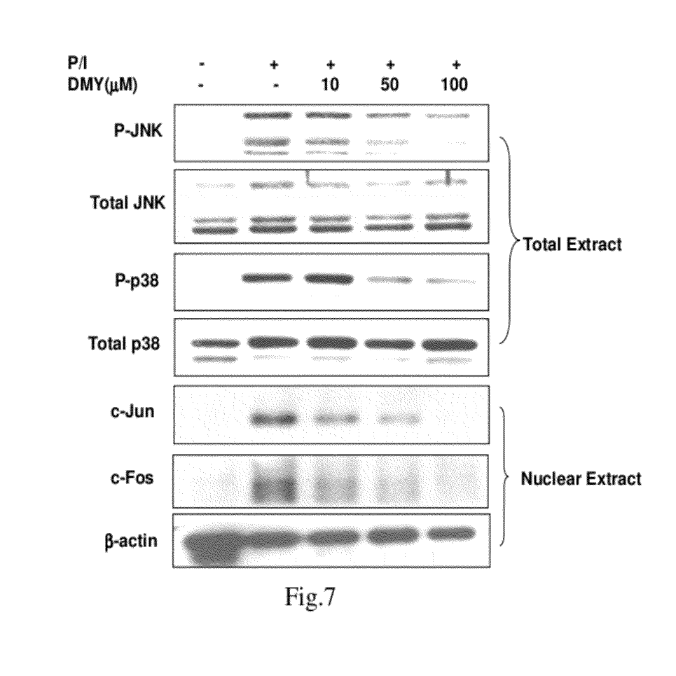

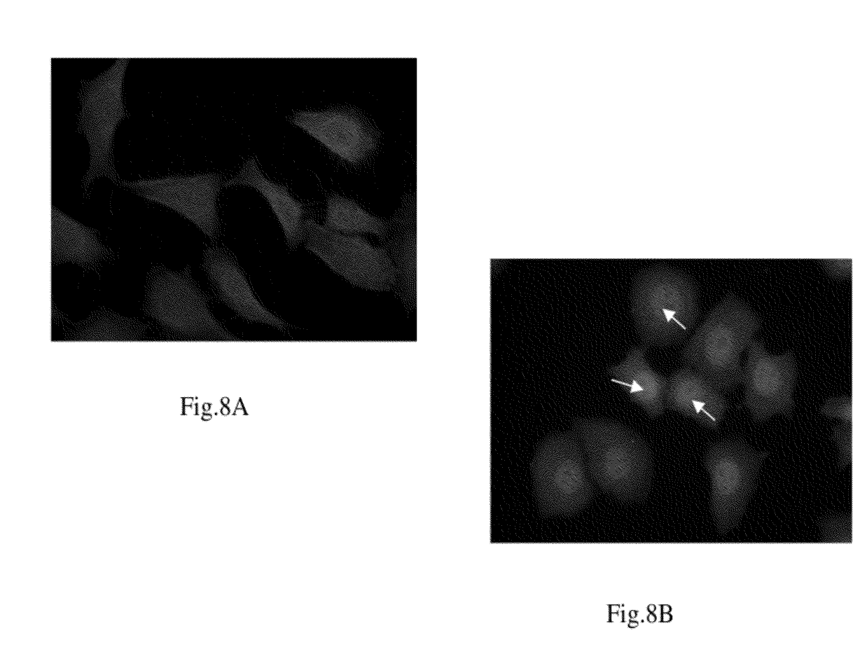

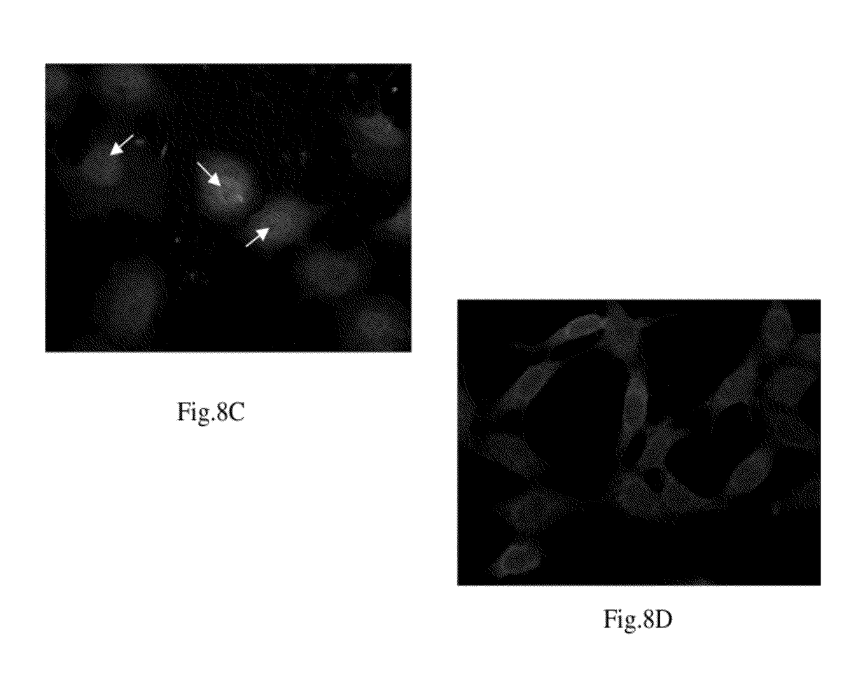

It can be observed from FIG. 7 that DMY suppressed p38 kinase and JNK phosphorylation. In addition, DMY is shown in FIGS. 8A to 8D to suppress nuclear translocation of c-Fos and c-Jun, the members of activated protein (AP)-1. In addition, DMY was shown to suppress c-Fos nuclear localization from the results of FIGS. 7 and 8. This implies that DMY suppressed AP-1 activation induced by PMA/Ionomycin. Thus in accordance with the present invention, DMY is also shown in FIG. 7 to have specific medical uses for the treatment of neurodegenerative disease, such as Alzheimer's disease, Parkinson's disease, amyotrophic lateral sclerosis, ataxia telangiectasia, spinocerebellar atrophy, multiple sclerosis, and Huntington's chorea.

### Example 6

### Study on Oral Acute Toxicity

The example describes the assays to show the low oral acute toxicity of DMY.

6.1 The Study

ICR (Imprinting Control Region) mice (both male and female) were purchased from the Laboratory Animal Services Center, the Chinese University of Hong Kong, Hong Kong. At the beginning of the study, one mouse was given orally with DMY at a dose of 2,000 mg/kg of mouse after fasting for 12 hours. The mouse had free access to water and after 2 hours, it was supplied with chow diet. Signs of toxicosis, onset of signs, and time to death of the mouse were monitored and recorded. Results of the initial exposure were used to select the subsequent dose of DMY, using the up-and-down method to estimate the lethal dose. If the mouse showed no signs of toxicosis upon receiving DMY, another two mice were exposed to the next dose of 5,000 mg/kg. If all the mice stayed alive, a higher dose of DMY was given to another three mice. All the mice were monitored continuously for two hours, every half hour for the next 5 hours, and at least every 10 hours until the 72nd hour of the study. Mice that died were immediately undergone autopsy. Mice remaining alive for 14 days upon study were sacrificed with an overdose pentobarbital.

6.2 Results

No toxic signs were observed at the dose of 2,000 mg/kg. For the five mice exposed to DMY at a dose of 5,000 mg/kg, three of them stayed still upon receiving the chemical but resumed to its normal state half an hour later, whereas the remaining two mice behaved normally. No clinical signs were observed within the next two weeks. So the acute oral LD₅₀ of DMY is greater than 5,000 mg/kg body weight for mice and it was thus considered practically non-toxic, and suitable for use as an oral medication. Potential usable dose ranges include 0.01 mg to 100 mg/kg, 0.01 to 50 mg/kg or 0.01 to 10 mg/kg in humans depending on the condition of the patient.

### Example 7

### Study on Ear EDEMA

The example describes the assays to show that topically application of DMY is effective to relief mouse ear edema.

7.1 The Delay-Type Hypersensitivity Test (DTHT) in Mice

Male ICR mice, weighting 22-30 g, were obtained from the Laboratory Animal Services Center, the Chinese University of Hong Kong (Hong Kong, China). Male mice were sensitized through topical application of 20 μl of 0.5% (v/v) dinitrofluorobenzene (DNFB) in acetone onto the shaved abdomen on days 1 and 2. Challenge was then preformed in day 6 by applying DNFB (20 μl, 0.5%, v/v) on the left inner and outer ear surfaces of mice. DMY (at doses of 0.5, 1, 2 mg/ear) and DEX (0.025 mg/ear, Sigma-Aldrich) dissolved in acetone was topically applied (20 μl) to the ears at 2nd, 24th, 48th, and 72nd hour after the challenge. The mice were sacrificed by cervical dislocation, and then the same area of the ears was punched from each animal. Spleens and thymuses were isolated and weighted. The ear edema was calculated according to the differences between the weight of the right and left ears. The control group was treated only with DNFB.

7.2 Results

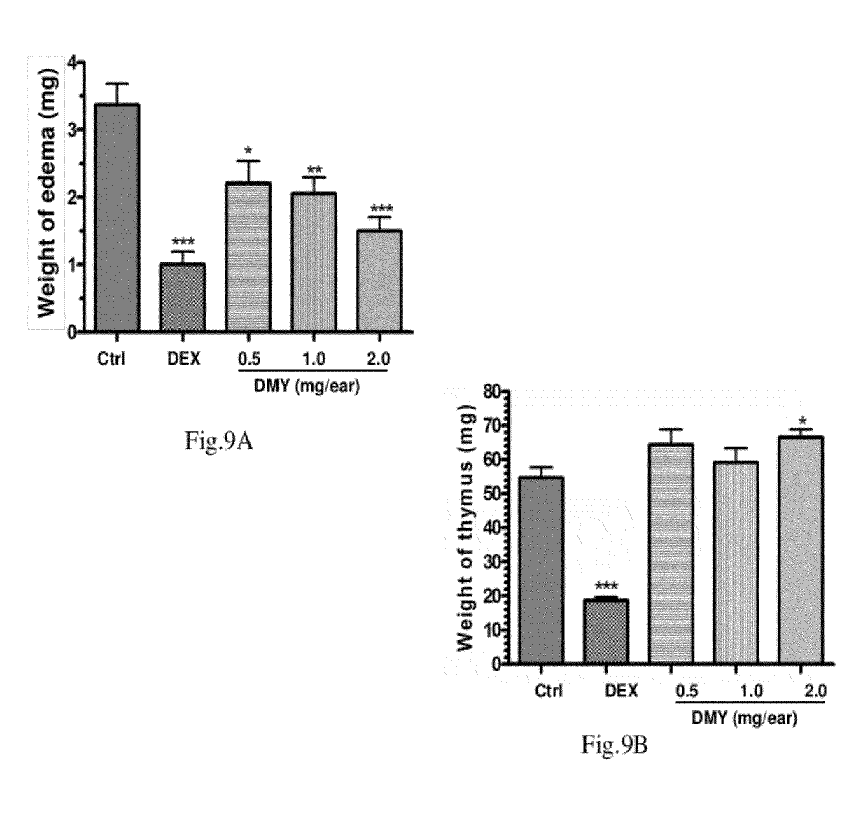

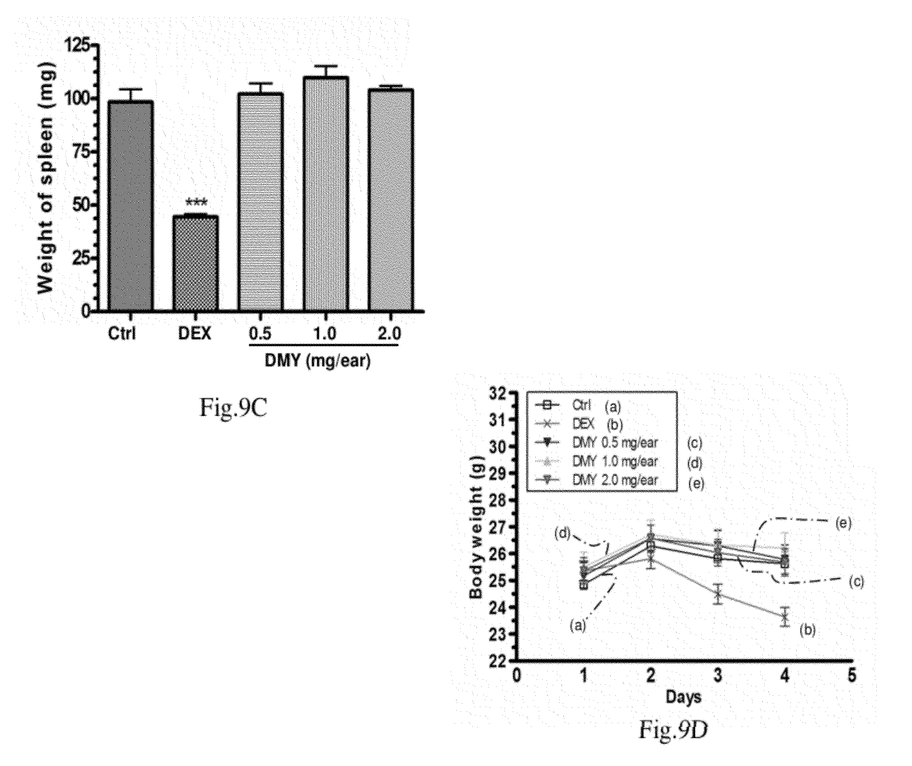

The DTHT test is the reaction triggered by antigen-specific T cells that can be induced by different allergens. In this study, the most commonly used allergen, DNFB which can effectively induce the contact dermatitis on ears was used. As observed from FIG. 9A, DMY could significantly and dose-dependently inhibit the ear edema of mice and the inhibition induced by of DMY is similar to the effect of DEX.

Besides, from FIGS. 9B and C spleen and thymus weights of the mice were decreased for DEX treatment, whereas an increase of weights of spleen and thymus can be observed for DMY treatment. Further, the body weight of the mice was greatly reduced for DEX treatment, while only a small decrease of body weight can be observed for mice treated with DMY in which the differences between body weights of mice in DMY treatment group and the control group were not significant.

In view of the above results, DMY suppresses hypersensitivity reaction of mouse ear edema induced by DNFB. DMY is also proven to be efficacious for the treatment of dermatitis, ear inflammation, and general inflammation, without adverse effect of general immunity suppression.

### Example 8

### Study on Arthritis

This example describes the study to show that DMY is effective to ameliorate collagen II induced arthritis in rats.

8.1 The Collagen II Induced Arthritis (CIA) in Rats

Female Wistar rats, 5-6 weeks old, were obtained from the Laboratory Animal Services Center, the Chinese University of Hong Kong (Hong Kong, China). Collagen II solution (collagen, 2 mg/ml in 0.05M acetic acid, Chondrex 20022, Redmond, Wash., USA) was emulsified with an equal volume of incomplete Freund's adjuvant (IFA, Chondrex 7002, Redmond, Wash., USA) at 4° C. using a high-speed homogenizer. In the experiment of CIA, DMY was encapsulated with HP-CD (1:8.48) and then dissolved in the normal saline with drug concentrations of 50 and 100 mg/kg body weight. Rats were intradermally injected at the base of the tail with 100 μl collagen/incomplete Freund's adjuvant (IFA) emulsion containing 100 g of collagen II by the use of a glass syringe equipped with a locking hub and a 27-G needle. On day 7 after the primary immunization, all the rats were given a booster injection of 100 μg of collagen II in IFA. On the day after the onset of arthritis (day 13), the CIA rats were exposed to a daily intraperitoneal administration of DMY (50 and 100 mg/kg) until day 30 of the study. DEX (0.1 mg/kg, one per day), MTX (3.75 mg/kg, twice per week), and indomethacin (1 mg/kg, one per day) were used as positive reference drugs.

The rats were inspected daily from the onset of arthritis characterized by edema and/or erythema in the paws. The incidence and severity of arthritis were evaluated using an arthritic scoring system, and bi-hind paw volumes and body weight were measured every 2 days started on the day when the arthritic signs were firstly visible (day 13). In the arthritic scoring system, lesions (i.e., the clinical arthritic signs) of the four paws of each rat were graded from 0 to 4 according to the extent of both edema and erythema of the periarticular tissues. As such, 16 was the potential maximum of the combined arthritic scores per animal. The hind paw volumes were measured using a plethysmometer chamber (7140 UGO. Basile, Comerio, Italy) and expressed as the mean volume change of both hind paws of the rats. Body weight of the rats was monitored with a 0.1 g precision balance (Sartorius AG, Goettingen, Germany). On day 30, all rats were sacrificed with liver, spleen and thymus being collected and weighted. The organ index for a specific organ is equal to the ratio of the weight of that organ to a body weight of 100 g.

8.2 Results

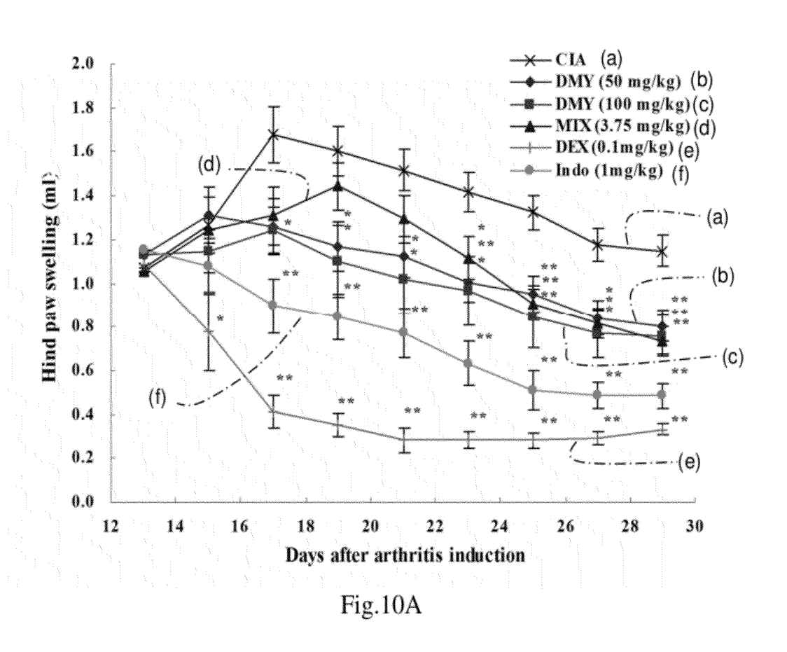

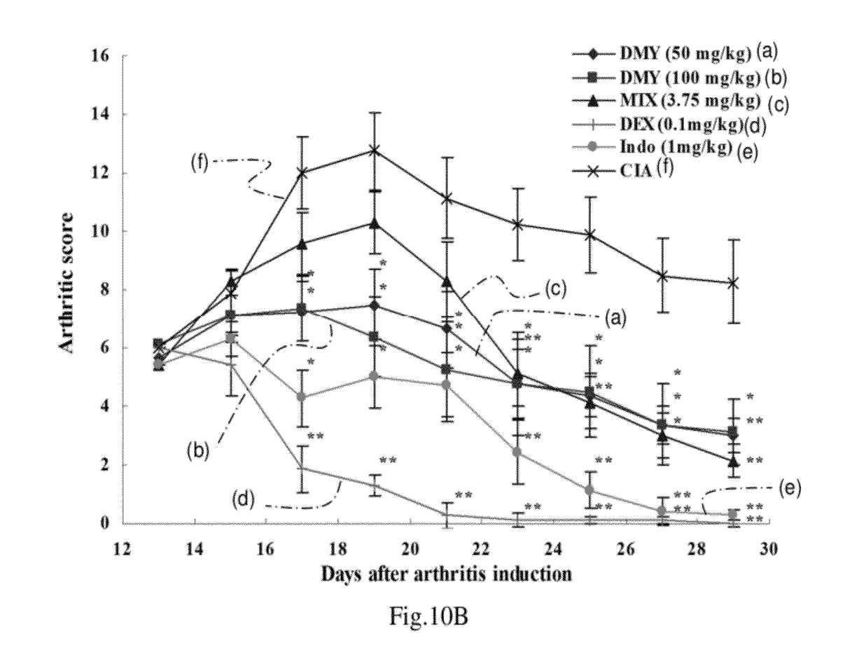

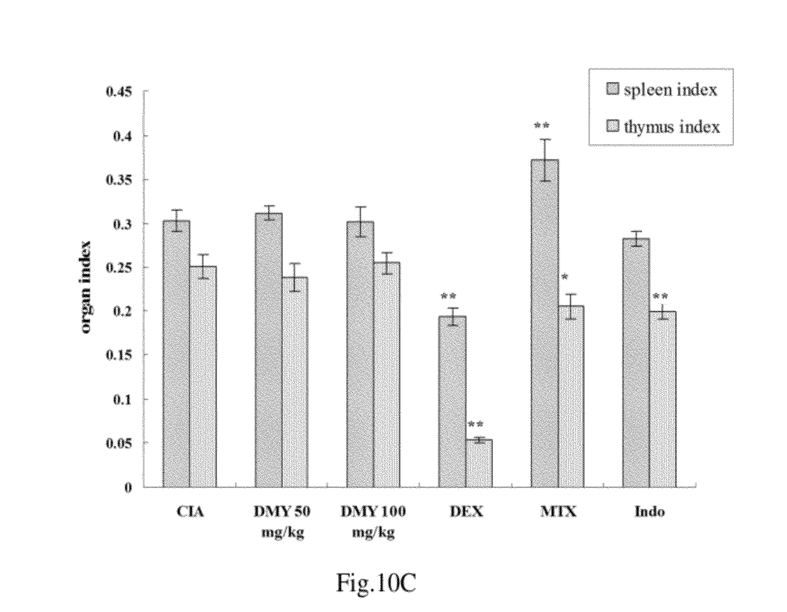

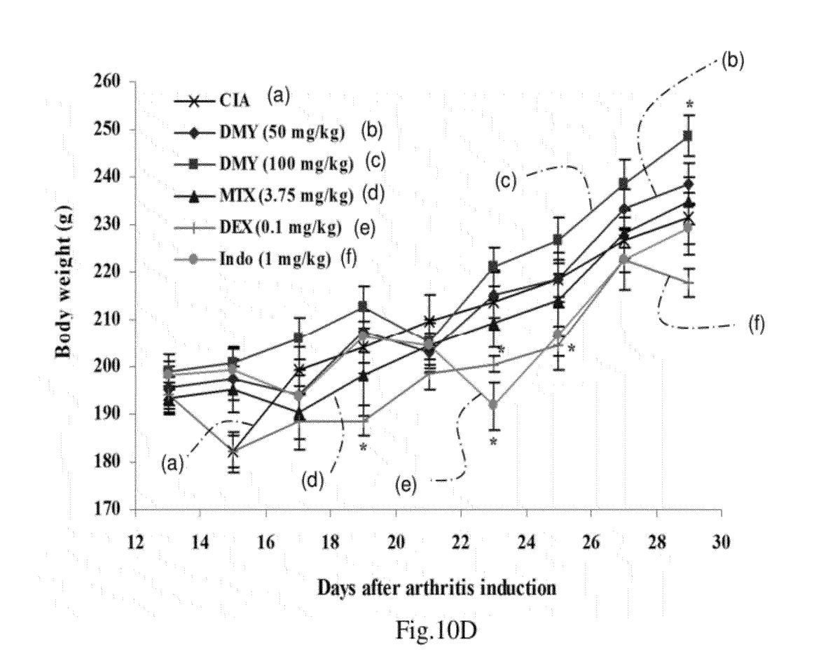

From FIGS. 10A and B, DMY treatment significantly reduced both the hind paw volume and the arthritic scores as compared to those of the vehicle-treated CIA rats, and the ameliorative effect of DMY at dose of 100 mg/kg (equivalent to human dose 16 mg/kg) was shown to be better than that of MTX. More importantly, it can be seen from FIG. 10C that there was no adverse effect on the organ indexes of spleen and thymus for DMY treatment, whereas treatments with DEX, MTX, or indomethacin led to a significant reduction of the organ indexes of spleen and/or thymus. In addition, a significant reduction in body weight can be observed for DEX-, MTX-, or indomethacin-treated animals from FIG. 100D, while the DMY-treated rats were shown even to have increase of the body weight.

In view of the above results, DMY suppresses arthritis induced by collagen II in rats. DMY is also proven to be efficacious for the treatment of arthritis and thus inflammation without adverse effect of general immunity suppression. The use of DMY is as described in the previous example.

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

For example, the pharmaceutical composition may be taken orally in different forms such as powder, capsule, or liquid.

### REFERENCES

1. Keifer J A, Guttridge D C, Ashburner B P, Baldwin A S, Jr. Inhibition of NF-kappa B activity by thalidomide through suppression of IkappaB kinase activity. J Biol Chem 2001; 276(25):22382-7.
2. Rossi A, Kapahi P, Natoli G, et al. Anti-inflammatory cyclopentenone prostaglandins are direct inhibitors of IkappaB kinase. Nature 2000; 403(6765): 103-8.
3. Hammaker D, Sweeney S, Firestein G S. Signal transduction networks in rheumatoid arthritis. Ann Rheum Dis 2003; 62 Suppl 2:ii86-9.
4. Castro A C, Dang L C, Soucy F, et al. Novel I KK inhibitors: beta-carbolines. Bioorg Med Chem Lett 2003; 13(14):2419-22.
5. Kishore N, Sommers C, Mathialagan S, et al. A selective IKK-2 inhibitor blocks NF-kappa B-dependent gene expression in interleukin-1 beta-stimulated synovial fibroblasts. J Biol Chem 2003; 278(35):32861-71.
6. Nagashima K, Sasseville V G, Wen D, et al. Rapid TNFR1-dependent lymphocyte depletion in vivo with a selective chemical inhibitor of IKKbeta. Blood 2006; 107(11):4266-73.
7. Schopf L, Savinainen A, Anderson K, et al. IKKbeta inhibition protects against bone and cartilage destruction in a rat model of rheumatoid arthritis. Arthritis Rheum 2006; 54(10):3163-73.
8. Burke J R, Pattoli M A, Gregor K R, et al. BMS-345541 is a highly selective inhibitor of I kappa B kinase that binds at an allosteric site of the enzyme and blocks NF-kappa B-dependent transcription in mice. J Biol Chem 2003; 278(3):1450-6.
9. McIntyre K W, Shuster D J, Gillooly K M, et al. A highly selective inhibitor of I kappa B kinase, BMS-345541, blocks both joint inflammation and destruction in collagen-induced arthritis in mice. Arthritis Rheum 2003; 48(9):2652-9.
10. MacMaster J F, Dambach D M, Lee D B, et al. An inhibitor of IkappaB kinase, BMS-345541, blocks endothelial cell adhesion molecule expression and reduces the severity of dextran sulfate sodium-induced colitis in mice. Inflamm Res 2003; 52(12):508-11.
11. Karin M, Yamamoto Y, Wang Q M. The IKK NF-kappa B system: a treasure trove for drug development. Nat Rev Drug Discov 2004; 3(1):17-26.
12. Ziegelbauer K, Gantner F, Lukacs N W, et al. A selective novel low-molecular-weight inhibitor of IkappaB kinase-beta (IKK-beta) prevents pulmonary inflammation and shows broad anti-inflammatory activity. Br J Pharmacol 2005; 145(2): 178-92.
13. Bamborough P, Callahan J F, Christopher J A, et al. Progress towards the development of anti-inflammatory inhibitors of IKKbeta. Curr Top Med Chem 2009; 9(7):623-39.
14. Edwards M R, Bartlett N R, Clarke D, Birrell M, Belvisi M, Johnston S L. Targeting the NF-kappaB pathway in asthma and chronic obstructive pulmonary disease. Pharmacol Ther 2009; 121(1):1-13.
15. Gagliardo R, Chanez P, Mathieu M, et al. Persistent activation of nuclear factor-kappaB signaling pathway in severe uncontrolled asthma. Am J Respir Crit Care Med 2003; 168(10):1190-8.
16. La Grutta S, Gagliardo R, Mirabella F, et al. Clinical and biological heterogeneity in children with moderate asthma. Am J Respir Crit Care Med 2003; 167(11): 1490-5.
17. Caramori G, Romagnoli M, Casolari P, et al. Nuclear localisation of p65 in sputum macrophages but not in sputum neutrophils during COPD exacerbations. Thorax 2003; 58(4):348-51.
18. Di Stefano A, Caramori G, Oates T, et al. Increased expression of nuclear factor-kappaB in bronchial biopsies from smokers and patients with COPD. Eur Respir J 2002; 20(3):556-63.
19. Sadikot R T, Zeng H, Joo M, et al. Targeted immunomodulation of the NF-kappaB pathway in airway epithelium impacts host defense against Pseudomonas aeruginosa. J Immunol 2006; 176(8):4923-30.
20. Podolin P L, Callahan J F, Bolognese B J, et al. Attenuation of murine collagen-induced arthritis by a novel, potent, selective small molecule inhibitor of IkappaB Kinase 2, TPCA-1 (2-[(aminocarbonyl)amino]-5-(4-fluorophenyl)-3-thiophenecarboxamide), occurs via reduction of proinflammatory cytokines and antigen-induced T cell Proliferation. J Pharmacol Exp Ther 2005; 312(1):373-81.
21. Hideshima T, Chauhan D, Richardson P, et al. NF-kappa B as a therapeutic target in multiple myeloma. J Biol Chem 2002; 277(19):16639-47.
22. Wen D, Nong Y, Morgan J G, et al. A selective small molecule IkappaB Kinase beta inhibitor blocks nuclear factor kappaB-mediated inflammatory responses in human fibroblast-like synoviocytes, chondrocytes, and mast cells. J Pharmacol Exp Ther 2006; 317(3):989-1001.
23. Onai Y, Suzuki J, Kakuta T, et al. Inhibition of IkappaB phosphorylation in cardiomyocytes attenuates myocardial ischemia/reperfusion injury. Cardiovasc Res 2004; 63(1):51-9.
24. Pierce J W, Schoenleber R, Jesmok G, et al. Novel inhibitors of cytokine-induced IkappaBalpha phosphorylation and endothelial cell adhesion molecule expression show anti-inflammatory effects in vivo. J Biol Chem 1997; 272(34):21096-103.
25. Frelin C, Imbert V, Griessinger E, Loubat A, Dreano M, Peyron J F. AS602868, a pharmacological inhibitor of IKK2, reveals the apoptotic potential of TNF-alpha in Jurkat leukemic cells. Oncogene 2003; 22(50):8187-94.
26. Birrell M A, Hardaker E, Wong S, et al. Ikappa-B kinase-2 inhibitor blocks inflammation in human airway smooth muscle and a rat model of asthma. Am J Respir Crit. Care Med 2005; 172(8):962-71.
27. Chapoval S P, Al-Garawi A, Lora J M, et al. Inhibition of NF-kappaB activation reduces the tissue effects of transgenic IL-13. J Immunol 2007; 179(10):7030-41.
28. Hu M C, Lee D F, Xia W, et al. IkappaB kinase promotes tumorigenesis through inhibition of forkhead FOXO3a. Cell 2004; 117(2):225-37.
29. Gringhuis S I, Garcia-Vallejo J J, van Het H of B, van Dijk W. Convergent actions of I kappa B kinase beta and protein kinase C delta modulate mRNA stability through phosphorylation of 14-3-3 beta complexed with tristetraprolin. Mol Cell Biol 2005; 25(15):6454-63.
30. Lee S, Andrieu C, Saltel F, et al. IkappaB kinase beta phosphorylates Dokl serines in response to TNF, IL-1, or gamma radiation. Proc Natl Acad Sci USA 2004; 101(50):17416-21.
31. Lee D F, Kuo H P, Chen C T, et al. IKK beta suppression of TSC1 links inflammation and tumor angiogenesis via the mTOR pathway. Cell 2007; 130(3):440-55.
32. Xia Y, Padre R C, De Mendoza T H, Bottero V, Tergaonkar V B, Verma I M. Phosphorylation of p53 by IkappaB kinase 2 promotes its degradation by beta-TrCP. Proc Natl Acad Sci USA 2009; 106(8):2629-34.
33. Yin M J, Yamamoto Y, Gaynor R B. The anti-inflammatory agents aspirin and salicylate inhibit the activity of I(kappa)B kinase-beta. Nature 1998; 396(6706):77-80.
34. Yamamoto Y, Yin M J, Lin K M, Gaynor R B. Sulindac inhibits activation of the NF-kappaB pathway. J Biol Chem 1999; 274(38):27307-14.
35. Waxman S, Anderson K C. History of the development of arsenic derivatives in cancer therapy. Oncologist 2001; 6 Suppl 2:3-10.
36. Kapahi P, Takahashi T, Natoli G, et al. Inhibition of NF-kappa B activation by arsenite through reaction with a critical cysteine in the activation loop of Ikappa B kinase. Biol Chem 2000; 275(46):36062-6.
37. Reynaert N L, van der Vliet A, Guala A S, et al. Dynamic redox control of NF-kappaB through glutaredoxin-regulated S-glutathionylation of inhibitory kappaB kinase beta. Proc Natl Acad Sci USA 2006; 103(35):13086-91.
38. Olsen L S, Hjarnaa P J, Latini S, et al. Anticancer agent CHS 828 suppresses nuclear factor-kappa B activity in cancer cells through downregulation of IKK activity. Int J Cancer 2004; 111(2): 198-205.
39. Gao Z, Hwang D, Bataille F, et al. Serine phosphorylation of insulin receptor substrate 1 by inhibitor kappa B kinase complex. J Biol Chem 2002; 277(50):48115-21.
40. Nakamori Y, Emoto M, Fukuda N, et al. Myosin motor Myolc and its receptor NEMO/IKK-gamma promote TNF-alpha-induced serine-307 phosphorylation of IRS-1. J Cell Biol 2006; 173(5):665-71.
41. Kamon J, Yamauchi T, Muto S, et al. A novel IKKbeta inhibitor stimulates adiponectin levels and ameliorates obesity-linked insulin resistance. Biochem Biophys Res Commun 2004; 323(1):242-8.
42. Yuan M, Konstantopoulos N, Lee J, et al. Reversal of obesity- and diet-induced insulin resistance with salicylates or targeted disruption of Ikkbeta. Science 2001; 293(5535):1673-7.
43. Thomas H and Waetzig V. AP-1 proteins in the adult brain: facts and fiction about effectors of neuroprotection and neurodegeneration. Oncogene 2001; 20:2424-37.
44. Buggia-Prevot V, Sevalle J, Rossner S, Checker F. NFkappaB-dependent control of BACE1 promoter transactivation by Abeta42. J Biol Chem. 2008; 283(15):10037-47.

What is claimed is:

1. A method of treating auto-immune disease, rheumatoid arthritis, chronic obstructive pulmonary disease (COPD), asthma, cancer, diabetes mellitus, neurodegenerative disease, immunological disorder, or arthritic disorder comprising administering an effective amount of dihydromyricetin (DMY) of formula (I).

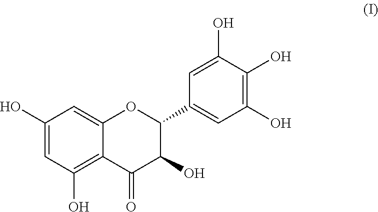

2. The method according to claim 1 wherein said neurodegenerative disease is selected from a group consisting of Alzheimer's disease, Parkinson's disease, amyotrophic lateral sclerosis, ataxia telangiectasia, spinocerebellar atrophy, multiple sclerosis, and Huntington's chorea.

3. The method according to claim 1 wherein said immunological disorder is selected from a group consisting of allergic rhinitis, allergic dermatitis, allergic contact dermatitis, allergic shock, asthma, papular urticaria, leucoderma, hypersensitivity vasculitis, hypersensitivity pneumonia, ulcerative colitis, glomerulonephritis, drug rashes, systemic lupus erythematosus, rheumatoid arthritis, scleroderma, multiple sclerosis, hyperthyroidism, idiopathic thrombocytopenic, autoimmune hemolytic anemia, allograft rejection, and hemolytic transfusion reaction.

4. The method according to claim 1 wherein said arthritic disorder is selected from a group consisting of rheumatoid arthritis, ankylosing spondylitis, gout, periarthritis, osteoarthritis, Reiter syndrome, psoriatic arthritis, post-traumatic arthritis, and enteropathic arthritis.

5. The method of treatment of claim 1 wherein said DMY is administered at a concentration of 0.1-100 mg/kg.

6. A pharmaceutical composition comprising DMY admixed with a pharmaceutical carrier suitable for use by oral administration.
